(19) United States
(12) Patent Application Publication
(10) Pub. No.: US 2008/0103489 A1
(43) Pub. Date: May 1, 2008

(54) Vacuum adherent dressings, systems and methods of use for same

(75) Inventor: Laurence E. Dahners, Chapel Hill, NC (US)

Correspondence Address: JENKINS, WILSON, TAYLOR & HUNT, P. A., 3100 TOWER BLVD., Suite 1200, DURHAM, NC, 27707, US

(73) Assignee: The University of North Carolina at Chapel Hill

(21) Appl. No.: 11/903,506

(22) Filed: Sep. 21, 2007

Related U.S. Application Data
(60) Provisional application No. 60/854,546, filed on Oct. 26, 2006.

(51) Int. Cl. A61M 27/00

(52) U.S. Cl. 604/543; 602/43

(57) ABSTRACT

Vacuum adherent dressings and methods and systems for using the vacuum adherent dressing are disclosed. The vacuum adherent dressing is for wound therapy of a wound having wound edges. The vacuum adherent dressing includes a flexible membrane having a top surface, a bottom surface, and a periphery. The membrane is configured to extend over the wound with the periphery of the membrane being distal from the wound edges. A fluid connector is provided on the membrane configured for attachment of a vacuum suction. Sealing jelly is disposed on the bottom surface of the membrane. The sealing jelly is configured to provide a seal between the membrane and a skin surface surrounding the wound up to and including the wound edges. One or more adhesive elements can be placed at one or more locations around the periphery of the membrane for positioning the membrane over the wound.

### RELATED APPLICATIONS

The presently disclosed subject matter claims the benefit of U.S. Provisional Patent Application Ser. No. 60/854,546, filed Oct. 26, 2006. the disclosure of which is incorporated herein by reference in its entirety.

### TECHNICAL FIELD

The subject matter described herein relates generally to vacuum adherent dressings. More particularly, the subject matter described herein relates to vacuum adherent dressings, systems and methods for their use which may be used over a wound that is open or closed that permit a less painful change of the dressings and provide more mobility for the patients on which the vacuum adherent dressings are used.

### BACKGROUND

Vacuum assisted healing of open wounds is a common practice today in many hospitals. The treatment of open wounds that are too large to spontaneously close has been a troublesome area for many years. In practice, a negative gauge pressure can be applied to a wound for a vacuum assisted closure. Vacuum assisted closure therapy typically involves the mechanical-like contraction of the wound with simultaneous removal of excess fluid. Such vacuum assisted closures and vacuum assisted closure therapies are described and detailed, for example, in U.S. Pat. Nos. 5,636,643 and 5,645,081 assigned to Wake Forest University, the disclosures of which are incorporated by reference herein in their entirety.

Wound closure requires that epithelial and subcutaneous tissue adjacent to the wound migrate toward and eventually close the wound. However, some wounds are sufficiently large or infected that they are unable to close spontaneously. In such instances, a zone of stasis, an area in which localized swelling of tissue restricts the flow of blood to these tissues, forms near the surface of the wound. This reduction in blood flow results in the wound being unable to successfully fight bacterial infection and prevents the spontaneous closure thereof.

By applying a negative pressure to the wound over the area sufficient to promote migration of the epithelial and subcutaneous tissue towards the wound, the closure of the wound is facilitated. The frequency at which negative pressure is applied to the wound, as well as the frequency of the pressure change over time, has a direct impact on the rate of the wound healing.

Often for these large wounds, a firm pad of open cell foam or other polymer material may be used to fill in the wound with a flexible polymer film overlying the foam section. A fluid connector or tubing is provided which penetrates the polymeric film sheet and engages the open cell foam to provide suction on the wound underneath the polymeric film sheet. The polymeric film sheet includes an adhesive which adheres to the skin that surrounds and extends into the wound area to provide a closed environment in which the suction from the tubing can create a vacuum surrounding the wound. The adhesive which seals the polymeric film to the skin extends adjacent to the edges of the badly injured tissue surrounding the wound and this tissue is usually very irritable.

Within a hospital, these vacuum assisted closure dressings must be changed on a regular basis, for example, every two or three days. To change the dressing, the polymeric film which is adhered to the skin with a strong adhesive must be ripped from the patient's skin. Pulling the adhesive off of the inflamed skin surrounding the wound can be excruciating to the patient. The pain can be so great that doctors often take patients back to the operating room and put them under an anesthetic before the vacuum assisted closure dressing is removed. Such steps to protect the patient are very time consuming and expensive.

Therefore a need exists for a vacuum adherent dressing that can be easily placed on open or closed wounds and that is not time consuming to apply and which can be easily removed without undue pain or discomfort to the patient.

### SUMMARY

In accordance with this disclosure, the subject matter provides novel vacuum adherent dressings, systems and methods that can be used to facilitate healing of both open and closed wounds.

It is therefore an object of the presently disclosed subject matter to provide vacuum adherent dressing devices, systems and methods that provide quick, easy and comfortable attachment of a vacuum adherent dressing to a patient's open or closed wound and also provide quick, easy and comfortable removal of the same dressing that can be tolerated by the patient on which they are placed. This and other objects as may become apparent from the present disclosure are achieved, at least in whole or in part, by the subject matter described herein.

### BRIEF DESCRIPTION OF THE DRAWINGS

A full and enabling disclosure of the present subject matter including the best mode thereof to one of ordinary skill in the art is set forth more particularly in the remainder of the specification, including reference to the accompanying figures, in which:

FIG. 1 illustrates a perspective view of an embodiment of a vacuum adherent wound dressing according to the present subject matter in use on an open wound;

FIG. 2 illustrates a cross-sectional view of another embodiment of a vacuum adherent dressing according to the subject matter described herein in use on an open wound;

FIG. 3 illustrates a perspective view of an embodiment of a vacuum adherent wound dressing according to the present subject matter described herein, used on a closed wound;

FIG. 4 illustrates a cross-sectional view of the embodiment of the vacuum adherent dressing according to FIG. 3; and

FIG. 5 illustrates an embodiment of a kit system with possible components that can be included in the kit system including embodiments of prefabricated, vacuum adherent dressings shown in cross sectional views.

### DETAILED DESCRIPTION

Reference will now be made in detail to the description of the present subject matter, one or more examples of which are shown in the figures. Each example is provided to explain the subject matter and not as a limitation. In fact, features illustrated or described as part of one embodiment can be used in another embodiment to yield still a further embodiment. It is intended that the present subject matter cover such modifications and variations.

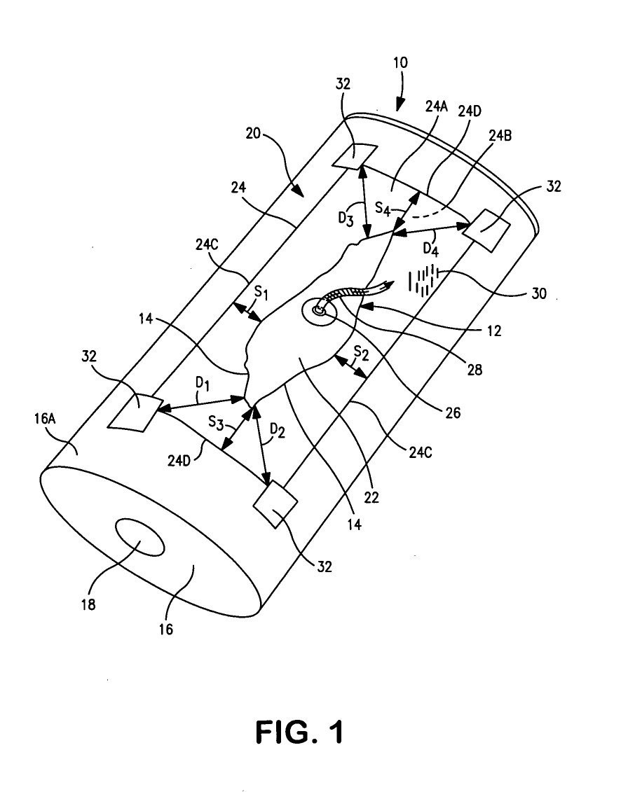

Referring to FIG. 1, a limb generally designated 10 is shown with a wound 12 defined by wound edges 14 within soft tissue 16. Wound 12 is an open wound that resides in soft tissue 16 around bone 18. A vacuum adherent dressing generally designated 20 has been placed in and over wound 12 to provide a vacuum assisted closure for wound 12. Vacuum adherent dressing 20 can include a porous dressing material such as a sponge 22 that can be placed within wound 12 between edges 14 of wound 12. Vacuum adherent dressing 20 further includes a flexible membrane 24 which has a top surface 24A and a bottom surface 24B. Membrane 24 can be a soft and thin thermoplastic film. For example, membrane 24 can comprise polyester, polypropylene, polyethylene, or the like. Plastic membrane 24 is occlusive enough to allow a creation of a vacuum over the wound with membrane 24 being sealed around wound 12.

A fluid connector 26 is provided on top surface 24A of plastic membrane 24. The term “fluid connector” as used herein includes a nipple, tubing, suction aperture or conduit that permits fluid flow through the membrane of vacuum adherent dressing. Such fluid connectors can be flat, cone-shape or be conduits or tubing that extend outward from the top surface and/or bottom surface of the membrane of the vacuum adherent dressing. Fluid connector 26 provides a connection point for suction tubing 28 that may be secured to a vacuum chamber or motorized device to provide a vacuum or suction on wound 12. In particular, fluid connector 26 can permit securement of suction tubing 28 around fluid connector 26 or tubing 28 can be inserted through fluid connector 26 and into sponge or gauze 22. Fluid connectors 26 can also be a tubing extending from top surface 24A that can be connected to a vacuum chamber or connected with an intermediate “Y” or multiple connector that is also connected to one or more other vacuum adherent dressings, for example, for patients with multiple wounds. The use of such an intermediate connector allows both or several vacuum adherent dressings to be attached to the same vacuum chamber. Alternatively or additionally, fluid connector 26 can extend outward from bottom surface 24B for insertion into the porous dressing material.

Bottom surface 24B of plastic membrane 24 may have a coating of sealing jelly 30 thereon. Sealing jelly 30 can cover bottom surface 24B of plastic membrane 24 up to and around fluid connector 26 and outward to the lengthwise and widthwise periphery 24C, 24D, respectively, of plastic membrane 24. Sealing jelly 30 provides a nonadhesive seal between the membrane 24 and skin surface 16A which surrounds the wound 12.

This undercoating of sealing jelly 30 can be, for example, a petroleum jelly, a water-based jelly, glycerol, bio-acceptable grease, or the like. The sealing jelly could be impregnated with antibacterial agents such as bacitracin, iodophors, chlorhexidine or the like. Sealing jelly 30 provides an air tight seal between the soft, thin flexible plastic membrane 24 and skin surface 16A that surrounds open wound 12. In this manner, when a vacuum is applied through suction tubing 28 and fluid connector 26, an air tight vacuum is created around wound 12 due to the closed environment created between plastic membrane 24, sealing jelly 30 and surrounding skin surface 16A of wound 12 when vacuum suction is applied.

Different sealing jellies can be used to provide different benefits in helping to heal open wounds such as open wound 12 shown in FIG. 1. For example, petroleum jelly has been shown to accelerate wound healing especially on open wounds and cuts because it inhibits germs from getting into the wound through the seal created between the plastic membrane 24 and skin surface 16A. Also, it keeps the skin surrounding the injured area supple by preventing skin moisture from evaporating. Such benefits can increase the rate of contraction and epithelialization, helping the wound heal at a much faster rate.

Similarly, water-based jellies can be used to provide many of the same benefits. However, such water-based jellies and glycerols may be absorbed by the skin over time. Therefore, if such sealing jellies are used, then the dressings would have to be changed on a regular basis to preserve the seal between the vacuum adherent dressing 20 and skin surface 16A surrounding wound 12.

Besides providing good sealing between plastic membrane 24 and skin surface 16A, sealing jelly 30 also provides a more comfortable seal that permits easy and comfortable removal of the vacuum adherent dressing 20 which greatly minimizes the discomfort and pain previously associated with changing such dressings.

Due to the lubricating nature of the sealing jelly 30 used within the vacuum adherent dressing 20 and the lower coefficient of friction between plastic membrane 24 and skin surface 16A created by the sealing jelly 30, one or more adhesive elements 32 may be placed along lengthwise and widthwise outer edges 24C and 24D, respectively, of plastic membrane 24 to hold plastic membrane 24 in position over wound 12. Adhesive elements 32 can be any adhesive material that can hold membrane 24 in place. Adhesive elements 32 can be, for example, strips of tape or other adhesive material. For instance, one inch by one inch discrete sections of tape can be placed partially over top surface 24A of membrane 24 at the four corners of peripheral edges 24C, 24D and partially on skin surface 16A. Similarly, adhesive elements 32 can be adhesive material applied to discrete portions of the underside of the peripheral edges 24C, 24D of membrane 24. The discrete adhesive elements 32 can be isolated from one another as shown in FIG. 1 and not be continuous around the periphery of membrane 24. Alternatively, the adhesive elements 32 can be in close proximity to each other around the peripheral edges 24C, 24D of membrane 24.

The vacuum adherent dressing 20 through the vacuum provided by a vacuum chamber and the sealing jelly 30 adheres in a position surrounding the wound 12. Thus, adhesive elements 32 do not adhere the vacuum adherent dressing to the skin so that the vacuum can be created. Adhesive elements 32 only hold vacuum adherent dressing in place to keep vacuum adherent dressing 20 from sliding.

By not having to use adhesive to create the seal between plastic membrane 24 and skin surface 16A which surrounds wound 12, a minimal number of skin attachment elements, such as adhesive elements 32, needs to be used to hold the portion of plastic membrane 24 over wound 12 in a relatively constant position without having to extend adhesive around the entire periphery of plastic membrane 24. Further, the distance between where adhesive elements 32 are secured to skin surface 16A and the edges 14 of wound 12 can be greatly increased. In this manner, skin surface 16A where adhesive elements 32 are secured will be much less sensitive than the skin surface which is much closer and/or abuts the edges 14 of wound 12.

For example, FIG. 1 shows four adhesive elements 32 positioned at each of the four corners of plastic membrane 24. The size and placement of the plastic membrane 24 can be such that the portion of the adhesive elements 32 may be secured to skin surface 16A at a distance D₁, D₂, D₃, D₄, from the respective nearest edge 14 of wound 12 so that each adhesive element 32 is attached to a portion of the skin surface 16A that is at worst only minimally inflamed or irritated by wound 12. Further, due to the limited amount of adhesive which is adhered to skin surface 16A, irritation caused by removal of vacuum adherent dressing 20 is minimized. A minimal amount of adhesive is used at points outside areas of various irritation of the skin caused by wound 12. Thus, when the adhesive elements 32 are removed the patient feels much less discomfort than if all of the membrane had an undercoating of adhesive that went up to the edges 14 of wound 12. Further, when plastic membrane 24 is removed, sealing jelly 30 eliminates pulling on the skin and thus provides no discomfort to the patient.

In use, a plastic membrane 24 with a fluid connector 26 may be provided with a sealing jelly 30 undercoating on bottom surface 24B of plastic membrane 24. Using such a plastic membrane 24 with an undercoating of sealing jelly 30 can create a good seal even around wounds which have protruding devices such as orthopedic pins extending therefrom. A protective paper strip or backing may be applied to bottom surface 24B and sealing jelly 30 which can be pulled away before application on a wound. The soft, thin, flexible plastic membrane 24 can have a substantial size, for example, about 12 inches by about 18 inches. Plastic membrane 24 can be prefabricated to have a cone-shaped fluid connector or a suction tube fitting extending therefrom. A porous dressing material 22 may be placed in an open wound 12 and the backing on plastic membrane 24 may be peeled off to reveal bottom surface 24B and sealing jelly 30. The plastic membrane 24, before or after the peeling of the backing, can be cut or trimmed to an appropriate size to fit the wound 12. For example, plastic membrane 24 would be trimmed to have plastic membrane edges 24C, 24D that provide enough sealing distance, S₁, S₂, S₃, S₄, around the respective edges 24C, 24D to provide a stable and proper seal to allow a vacuum to be created around wound 12. At the same time, trimming the plastic membrane 24 can create distances D₁, D₂, D₃, and D₄ to ensure that the placement of any adhesive elements 32 is at a distance far enough away from edges 14 of wound 12 to minimize any discomfort associated with removal of these isolated adhesive elements 32.

The trimming of plastic membrane 24 should be done so as to take into account the proper placement of fluid connector 26 so that suction tubing 28 can maximize the vacuum created over wound 12. Again, the amount of skin attachment elements, such as adhesive elements 32, which is secured to skin surface 16A should be just enough to prevent undue sliding of plastic membrane 24 off of the wound or to a point where the vacuum created over the wound is obstructed. Further, it is noted that the size and trimming of plastic membrane 24 and the distances D₁, D₂, D₃, or D₄, as well as the sealing distances S₁, S₂, S₃, S₄ can be varied based on the severity of the wound, the position of the wound on the patient's body, the severity of the patient's condition, or the like. Such factors should be taken in to consideration when deciding on optimal sealing distances and optimal distances between the edge 14 of wound 12 and the placement of adhesive from adhesive element 32 on skin surface 16A.

Once it is time to change the dressing, the suction through suction tubing 28 can be stopped. Saline may be infused through suction tubing 28 and allowed to loosen the dressing where it has become adherent to the wound tissues 12 prior to removal. The adhesive elements 32 can then be removed from the specific locations where they had been placed to hold plastic membrane 24 in place. Alternatively, if the dressing is being changed rather than removed for good, plastic membrane 24 can be cut loose from adhesive elements 32 and adhesive elements 32 can be left in place on the skin. At this point, the vacuum adherent dressing 20 can be easily pulled off the skin with minimal patient discomfort. The sealing jelly 30 permits quick and relatively pain-free removal of plastic membrane 24 that breaks the seal when pulled in an upward fashion while at the same time permitting a solid seal when plastic membrane 24 is placed on skin surface 16A and a vacuum applied. When a new dressing is applied, new adhesive elements 32 can be applied on top of the old adhesive elements 32 so that the painful removal of adhesive elements need only occur after the wound is healed and dressing changes are no longer necessary (or when the adhesive fails and they spontaneously and painlessly disengage but must be replaced).

In this manner, a marked decrease in patient discomfort during dressing changes can occur. Further, by using sealing jelly 30 up to and around wound 12, wet or bloody skin or orthopedic pins are less of a concern in creating a seal between plastic membrane 24 and skin surface 16A because the sealing jelly fills into defects unlike when adhesive membranes are used to create such a seal. Also, when an adhesive membrane is removed it often is very difficult to remove from orthopedic pins due to its adhesion but membranes sealed with jelly can be easily removed. Further, if placement of vacuum adherent dressing 20 is difficult due to the position of the wound, sealing jelly 30 provided on the bottom surface 24B of plastic membrane 24 allows for easy repositioning and/or pulling off and reapplying of plastic membrane 24 to obtain optimum positioning of the vacuum adherent dressing 20. Such sealing jelly-undercoated plastic membranes also work well when applied near orthopedic external fixators without the membranes sticking to the frame and tearing due to the capability of the undercoated plastic membrane being easily repositioned and moved. Further, when sealing is made difficult by wet, bloody skin or some other external device such as orthopedic pins, additional sealing jelly that can be provided with the vacuum adherent dressing 20 in for example, a kit, would allow the application of extra sealant to seal any leaks.

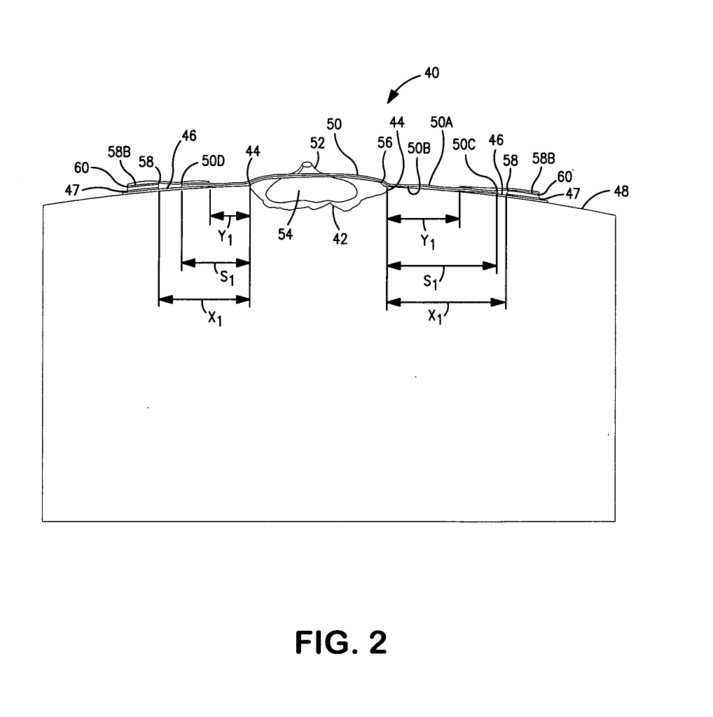

FIG. 2 illustrates another embodiment of a vacuum adherent dressing, generally designated as 40. Vacuum adherent dressing 40 can be used on wounds which would require vacuum assisted closure for an extended period of time, for example, multiple weeks. When a wound such as wound 42 having wound edges 44 is first being treated, an adhesive element 46 may be placed at different locations at a distance X₁ as measured from edges 44 of wound 42. Each adhesive element 46 can include a fastener portion 47 such as a hook or a loop portion of a hook and loop fastener that extends from an upper surface of the adhesive element 46 outward from the skin surface 48. The distance X₁ can be large enough such that adhesive element 46 is placed at a location where skin surface 48 is less irritable or inflamed due to the wound 42.

A membrane 50 having a top surface 50A and a bottom surface 50B can be provided. Plastic membrane 50 can include a fluid connector 52 which can be aligned with wound 42. As above, membrane 50 can be a thermoplastic film. Wound 42 can be an open wound in which a porous dressing material 54, such as sponge or gauze, can be placed. Bottom surface 50B can include an undercoating of sealing jelly 56 that extends outward to the edges 50C, 50D of membrane 50. As stated above, membrane 50 can be a thin, flexible soft membrane that limits air permeability to help to create a vacuum seal when fluid connector 52 is secured to a suction tubing of a vacuum chamber. An extension member 58 can be secured to top surface 50A of membrane 50. Extension members 58 can be aligned with adhesive elements 46 and can have a matching fastener portion 60 on underside 58B of extension member 58 that can be aligned and engaged with fastener portion 47 of adhesive element 46. For example, matching fastener portion 60 can be the other portion of the hook and loop fastener that mates with the hook and loop fastener of fastener portion 47. Fastener portion 47 and matching fastener portions 60 can be other fastening devices such as the male and female end of a snap, a button and button hole, or the like.

Sealing jelly 56 extends outward on bottom surface 50B of membrane 50 toward edges 50C, 50D for a distance S₁. Distance S₁ is large enough to create a seal between skin surface 48 and plastic membrane 50 so that, when a vacuum is applied through fluid connector 52, wound 42 and wound area surrounding it is adequately sealed to facilitate the creation of a vacuum therein. Sealing jelly 56 also provides an additional benefit of preventing the migration of germs or other bacteria into the wound 42 and increasing the epithelialization and contraction of wound 42. As described above, sealing jelly 56 can include antibacterial agents.

In the embodiment shown in FIG. 2, the vacuum adherent dressing, in particular membrane 50, can be easily removed by disengaging fastener portions 47, 60 from one another so that plastic membrane 50 can be removed while the adhesive elements 46 will remain in place. Plastic membrane 50 can then be discarded and a new membrane 50 can be applied over wound 42 with new porous dressing material 54, such as a sponge or gauze, placed within wound 42 such that fluid connector 52 is aligned to provide a vacuum suction over wound 42. New membrane 50 can again include an undercoating of sealing jelly 56 and have extension members 58 which provide matching fastener portions 60 that can be aligned with fastener portions 47 of the adhesive elements 46 that still reside on skin surface 48.

In this manner, a severe wound which takes several weeks to heal can have its vacuum adherent dressing assembly 40 changed with little or no discomfort to the patient in an easy and secure manner that provides adequate sealing for the creation of the necessary vacuum over wound 42. Since adhesive elements 46 may reside on skin surface 48 for several weeks, the wound may heal enough where removal of adhesive elements 46 does not cause extreme discomfort or excruciating pain for the patient. Thereby, adhesive elements 46 to ensure adequate securement to skin surface 48 may extend to a distance Y₁ from wound edges 44 which is closer to wound edges 44 of wound 42. By the time adhesive elements 46 need to be removed, the wound 42 may be close to fully healing and the removal of adhesive elements 46 may not be in close proximity to inflamed or irritated skin surrounding wound 48 due to the healing of the wound. In this manner, the vacuum adherent dressing 40 can be changed many times before adhesive element 46 needs to be removed from skin surface 48.

While the above embodiments may also be used on closed wounds as well as open wounds, other advantageous embodiments may be used in conjunction with closed wounds to facilitate easy changing of the dressing as well as provide mobility to the patient which is not readily available to patients which need to have their vacuum adherent dressing secured to a stationary vacuum pump device.

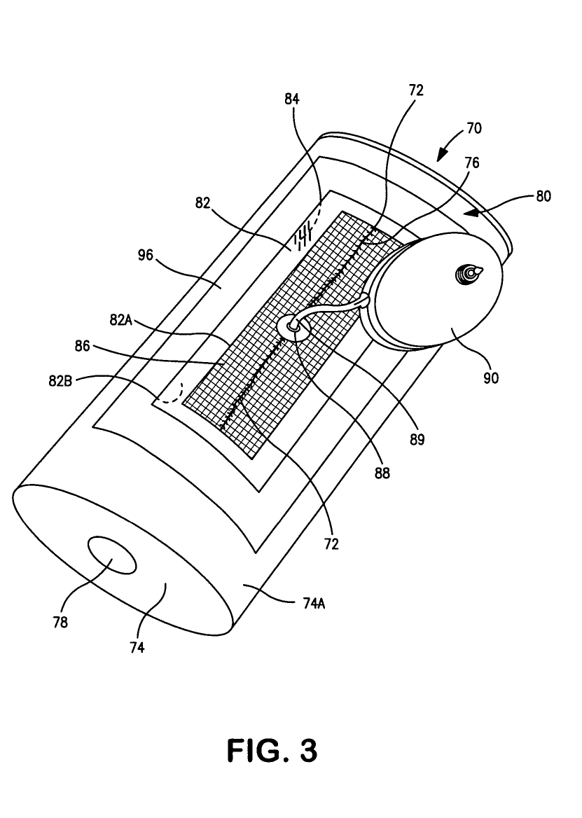

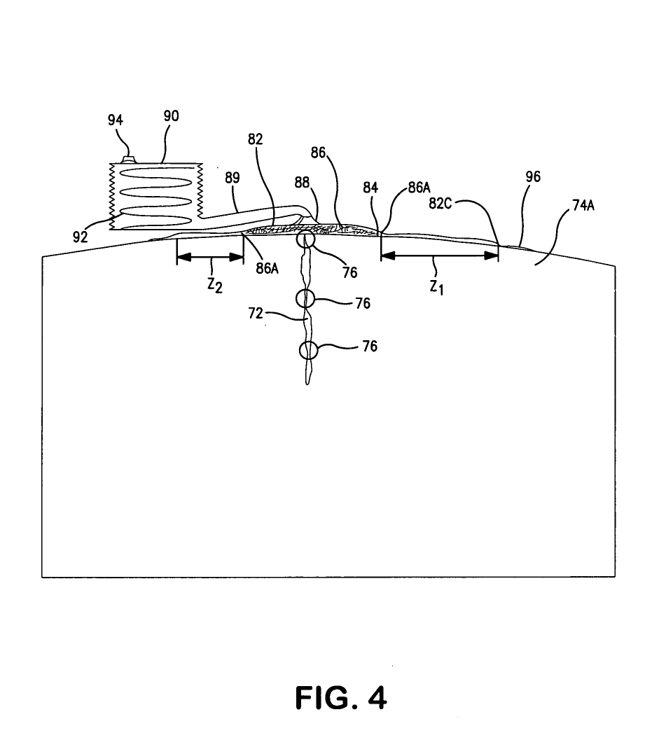

FIGS. 3 and 4 show a further embodiment of a vacuum adherent dressing. A limb generally designated 70 has a wound, or incision, 72 in soft tissue 74. Wound 72 has been closed by sutures 76 or other securing devices used to close wounds. Closed wound 72 which can be in the soft tissue 74 above bone 78 of limb 70 can have its healing facilitated by a vacuum adherent dressing generally designated 80. Vacuum adherent dressing 80 includes a plastic membrane 82 which has a top surface 82A and a bottom surface 82B. Plastic membrane 82 can be thin, flexible and soft and can be occlusive enough to permit creation of a vacuum over the wound when sealed against surrounding skin surface 74A. Plastic membrane 82 can have an undercoating on bottom side 82B that includes a sealing jelly 84. Gauze 86 can be extended over the top of wound 72 between sealing jelly 84 and skin surface 74A surrounding closed wound 72. A fluid connector 88 may reside on plastic membrane 82 that permits a fitting to a suction tubing 89 that is secured to a portable vacuum chamber or pump 90. Fluid connector 88 can be aligned in a central location of the plastic member 88 over closed wound 72 with gauze 86, or other air and liquid permeable dressing material, positioned underneath fluid connector 88. An adhesive element, or strip, 96 may be placed around the perimeter or periphery 82C (see FIG. 4) of plastic member 82 to secure the placement of the plastic member 82 to skin surface 74A at a distance away from wound 72 that permits more comfort during removal of vacuum adherent dressing 80 from skin surface 74A.

As described above, sealing jelly 84 creates a seal between plastic member 82 and skin surface 74A to allow portable vacuum chamber or pump 90 to create a sufficient vacuum around wound 72 to facilitate healing of the wound. Such vacuum does not have to be as great as the vacuum created by an automated stationary pump vacuum.

Portable vacuum chamber 90 can be a battery operated vacuum chamber which is secured to the limb on which the wound has occurred or to other parts of the patient. For example, a small pump that operates as portable vacuum chamber 90 and can be powered by AA batteries can be secured to and reside on the vacuum adherent dressing 80 or the limb of the patient. Alternatively, as shown in FIGS. 3 and 4, portable vacuum chamber 90 may be a simple manual spring load vacuum chamber that creates a low level vacuum over wound 72. As shown in FIG. 4, spring loaded vacuum chamber 90 can include a spring 92 and a closeable valve 94 that permits the spring loaded vacuum chamber to be compressed with the valve 94 then being closed. When the compression is released spring 92 pushes upward to create a vacuum through suction tubing 89 and fluid connector 88 over the top of wound 72 within gauze 86. As needed, the suction created through spring loaded vacuum chamber 90 can be repeated by the user. Such a spring loaded vacuum can be easily attached to the limb without being placed at some other location on the patient. Spring loaded vacuum chamber 90 can create a low grade or a high grade vacuum both of which can be helpful even on a closed wound 72 in facilitating healing.

As shown in FIG. 4, sealing jelly 84 can extend past gauze 86 outward to create a sufficient distance distances Z₁, Z₂ so that the position of the adhesive strip 96 at the periphery 82C of plastic membrane 82 is far enough away as to not irritate the skin around wound 72. Further, the distances Z₁, Z₂ which sealing jelly 84 extends from gauze 86 is enough to create a proper seal that allows spring loaded vacuum chamber 90 to create a vacuum over wound 72. Adhesive strip 96 can actually be a part of plastic membrane 82. The plastic membrane 82 can have a narrow strip of adhesive material applied to bottom surface 82B at the periphery 82C. In such embodiments, the sealing jelly 84 can extend up to an edge proximal to the narrow strip of adhesive material on bottom surface 82B.

To facilitate use, vacuum adherent dressing 80 can be prefabricated. Prefabricated vacuum adherent dressing 80 can include a plastic membrane 82 which is an occlusive membrane that eliminates air flow therethrough with an adhesive element 96 secured around the periphery or edge 82C to allow it to stick on and not shift or pull loose from skin surface 74A. The adhesive element 96 is preferably a narrow strip that is located far from the injured wound tissue, or incision, 72 so that it will hurt less if removed after several days. Membrane 82 further can include a fluid connector 88 which extends outward and can be secured to spring loaded vacuum chamber 90 that is placed in a middle portion of plastic occlusive member 82. A strip of gauze 86 is aligned underneath the connection of the suction tubing to the membrane 82. The strip of gauze 86, or other porous dressing material, can be aligned so that when placed on the wound it lies directly over the wound and allows for suction to extend over the length of the wound. At the edges 86A of gauze 86, an undercoating of sealing jelly 84 may extend outward toward the periphery or edge 82C of plastic membrane 82.

A backing can be secured on adhesive element, or strip, 96 so that it covers both sealing jelly 84 and gauze 86 to protect the vacuum adherent dressing 80 before it is applied to the patient. The backing can be peeled off of vacuum adherent dressing 80 before use in a similar fashion as backings on adhesive bandages. The vacuum adherent dressings 80 can come in different sizes to accommodate different sized wounds. Further, tape may be supplied with the prefabricated vacuum adherent dressing 80 so that periphery 82C of membrane 82 can be trimmed if necessary to fit closed wound 72. Thus, if adhesive element 96 is removed through a trimming down of plastic member 82 then the adhesive tape can be applied to secure the prefabricated vacuum adherent dressing 80 on limb 70. In such a case, distances Z₁, Z₂ may be shortened. However, distances Z₁, Z₂ can be maximized for the available size of the area at the location of the wound to maximize comfort for the patient.

Again, sealing jelly 84 on bottom surface 82B of plastic membrane 82 helps to achieve the vacuum seal and to prevent pulling on the periwound tissue when the dressing is removed. Also, in the prefabricated dressing 80, sealing jelly 84 can extend between membrane 82 and gauze 86 so that sealing jelly 84 can help to speed re-epithelialization of wound 72 so that the wound heals faster. By already having membrane 82 attached to tube 89 and spring loaded vacuum chamber 90, easy attachment and use can be performed to quickly facilitate healing of a closed wound, or incision, 72.

By using a plastic occlusive dressing or membrane, lower infection rates and less ingress of bacteria can be achieved. Suction applied to the wound through the spring loaded vacuum chamber can further improve healing even on the closed wound, or incision. The vacuum chamber can be used to collect any blood or other fluid leaking from the wound, thus keeping it from soiling the patient's clothing or bed clothes and thus lessening the chance that the patient or patient's family will be disturbed by such fluid leakage. When using such a prefabricated vacuum adherent dressing, sealing jelly under much of the dressing facilitates re-epithelialization of the wound and decreases the pain of the dressing removal. Further, the prefabricated vacuum adherent dressing provides a simple application for a surgeon by pulling it off a paper or other backing and applying the dressing over the wound. As described above, several different sizes of dressing for different sized wounds can be provided. An extra strip of adhesive membrane or tape can come in the package to allow the surgeon to trim the prefabricated dressing if necessary to fit the area in which the wound, or incision, has occurred and stick the cut edges down with the strip of the adhesive membrane. Since a low-grade vacuum can effectively be used with closed wounds, a much lower cost can be associated with the spring loaded vacuum chamber which is used in conjunction with the prefabricated vacuum adherent dressing described above.

Through the use of prefabricated vacuum adherent dressings, a vacuum adherent dressing system can be provided that allows more options for the surgeon or medical personnel applying the vacuum adherent dressing to the wound of a patient.

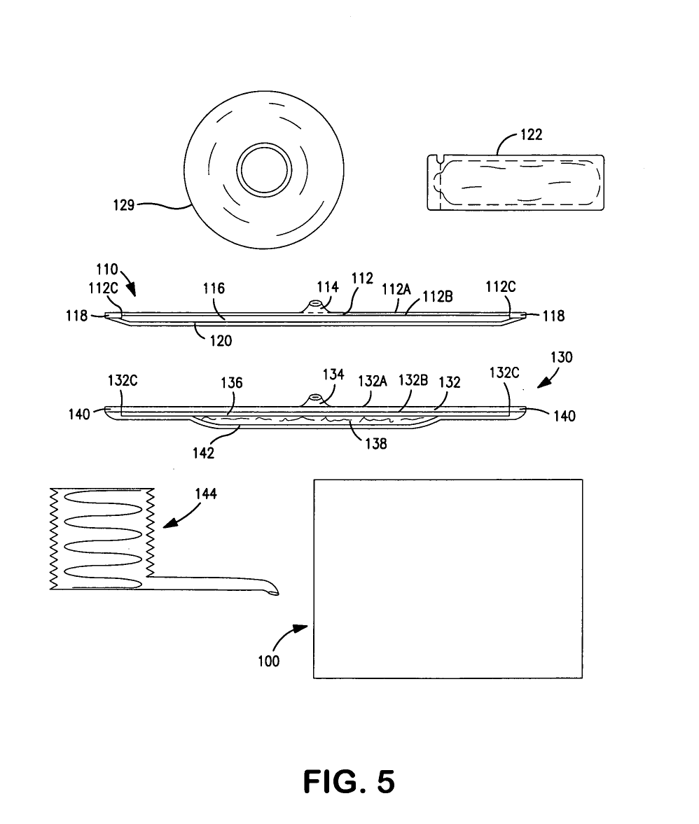

For example, FIG. 5 shows a kit 100 in schematic form. Depending on its use, kit system 100 can include a plurality of prefabricated vacuum adherent dressings. For example, prefabricated vacuum adherent dressings generally designated 110 and shown in cross-section can be provided which do not include gauze therein. The prefabricated vacuum adherent dressings 110 can include a soft, thin, flexible membrane 112 that includes a centralized fluid connector 114. Fluid connector 114 can be cone shaped or it can be flat and have a hole in which a suction tube can be inserted therethrough. Further, the suction tube can be integral with membrane 112 such that it can be inserted into a second suction tubing or directly into a vacuum chamber. Plastic membrane 112 further includes a top surface 112A and a bottom surface 112B on which an undercoating of sealing jelly 116 can reside. Extending out from membrane 112, the vacuum adherent dressing 110 can include adhesive elements 118 that are secured to a periphery 112C of membrane 112. A sheet of backing material 120, such as backing paper, can be secured to adhesive elements 118 and sufficiently cover bottom surface 112B of membrane 112 and sealing jelly 116 to prevent contamination thereof. The backing 120 can easily be peeled off to allow securement of vacuum adherent dressing 110 over a wound as necessary. As described above, adhesive elements 118 can comprise adhesive material applied to bottom surface 112B at periphery 112C of membrane 112.

As described above, different sized vacuum adherent dressings 110 can be included in kit system 100 to secure adequate fit on open and/or closed wounds as necessary. Packets of supplemental sealing jelly 122 can be included to ensure proper sealing in case of odd shaped areas on which the vacuum adherent dressing 110 must be placed. Packages of supplemental tape 124 or other adhesive elements can also be included in kit system 100 in case trimming of membrane 112 needs to occur to ensure proper fit over the wound area on which vacuum adherent dressing 110 is placed. Kit system 100 can also contain porous dressing material, such as sponges or gauze, that can be used in an open wound or on a closed wound in conjunction with or as a part of vacuum adherent dressing 110.

Similarly, kit system 100 can include a prefabricated vacuum adherent dressing 130 which can be used on a closed wound. As shown in the cross-sectional view, vacuum adherent dressing 130 can include a membrane 132 which is constructed of a soft flexible and thin material that further has a top surface 132A and a bottom surface 132B. Membrane 132 can include a fluid connector 134 on top surface 132A which permits connection to a vacuum chamber to create a vacuum over the wound once vacuum adherent dressing 132 is placed on a patient's wound. A layer of sealing jelly 136 can be secured to the bottom surface 132B of membrane 132. A porous dressing material 138 such as gauze can then be placed at a central location underneath fluid connector 134 without extending the full width or length of membrane 132 so that both membrane 132 and sealing jelly 136 extend outward from gauze 138. Sealing jelly 136 can extend between membrane 132 and gauze 138. Alternatively, sealing jelly 136 can extend beneath gauze 138, so that sealing jelly 136 is against the wound. In such an embodiment, an adhesive on membrane 132 can held gauze 138 in place. One or more adhesive elements 140 can be secured to periphery 132C of membrane 132. As described above, adhesive element 140 can comprise adhesive material applied to bottom surface 132B at periphery 132C of membrane 132. Backing material 142 can then be secured to the adhesive elements 140 and the layer of sealing jelly 136 that extends outward from gauze 138. In this manner, backing material 142 can completely cover the bottom of the vacuum adherent dressing 132 to keel) it sterile and ready for use.

Alternatively, prefabricated vacuum adherent dressing 130 can have soft, thin, flexible membrane 132 with a top surface 132A and a bottom surface 132B with adhesive along the bottom surface 132B of the flexible membrane 132 with no sealing jelly 136 disposed thereon. A porous dressing material 138 such as gauze can then be placed at a central location underneath fluid connector 134. In such an embodiment, the prefabricated vacuum adherent dressing 130 would be sealed over the wound by the adhesive.

In another alternative embodiment, porous dressing material 138 can be impregnated with sealing jelly 136, for example on its undersurface and heavily around its periphery. Soft, thin, flexible membrane 132 with a top surface 132A and a bottom surface 132B can have adhesive along the bottom surface 132B of the flexible membrane 132 with no sealing jelly 136 disposed thereon. The side of porous dressing material 138 with no sealing jelly 136 can be secured to the adhesive on membrane 132. The periphery of porous dressing material 138 can be heavily loaded with sealing jelly 136 to keep the vacuum from breaking through. Then, backing material 142 can be applied next. The area of membrane 132 around the periphery that has adhesive exposed can extend out beyond the jelly coated porous dressing material 138 by about ½ inch wide. Thus, the jelly impregnated porous dressing material 138 provides the non stick area of the dressing and the jelly seal.

A plurality of such vacuum adherent dressings 130 can be included in a separate kit system 100 or in the same system as vacuum adherent dressings 110. The plurality of vacuum adherent dressings 130 can come in different sizes to accommodate different sized wounds. As stated above, additional packages of supplemental sealing jelly 122 and package of supplemental adhesive elements 124 can be included in kit 100 to allow for the trimming of any of the vacuum adherent dressings 130 as needed. Further, extra porous dressing material, such as gauze or sponges, can be included.

For use with the vacuum adherent dressing 130, a vacuum chamber and suction tubing 144 can be included in the kit 100 for attachment to fluid connector 134. Alternatively, each dressing 130 can have an integral vacuum chamber 144 attached to membrane 132 to provide a complete package that is disposable after use.

Embodiments of the present disclosure shown in the drawings and described above are exemplary of numerous embodiments that can be made within the scope of the appending claims. It is contemplated that the configurations of the vacuum adherent dressing, systems and methods can comprise numerous configurations other than those specifically disclosed. The scope of a patent issuing from this disclosure will be defined by these pending claims.

What is claimed is:

1. A vacuum adherent dressing for wound therapy of a wound having wound edges, the vacuum adherent dressing comprising:
(a) a flexible membrane having a top surface, a bottom surface, and a periphery, the membrane configured to extend over the wound with the periphery of the membrane being distal from the wound edges;
(b) a fluid connector provided on the membrane configured for attachment of a vacuum suction;
(c) sealing jelly disposed on the bottom surface of the membrane, the sealing jelly configured to provide a seal between the membrane and a skin surface surrounding the wound up to and including the wound edges; and
(d) one or more skin attachment elements at one or more locations around the periphery of the membrane for positioning the membrane over the wound.

2. A vacuum adherent dressing according to claim 1 wherein the periphery of the membrane is at a distance from the wound edge to create a vacuum seal between the skin surface surrounding the wound and sealing jelly without use of an adhesive.

3. The vacuum adherent dressing according to claim 1 wherein the membrane comprises a thermoplastic film.

4. The vacuum adherent dressing according to claim 1 wherein the sealing jelly comprises at least one of a petroleum jelly, a water-based jelly or a bio-acceptable grease.

5. The vacuum adherent dressing according to claim 1 wherein the sealing jelly is impregnated with an antibacterial agent.

6. The vacuum adherent dressing according to claim 1 wherein the one or more skin attachment elements are positioned around the periphery of the membrane at a distance from the wound edge that minimizes irritation of the wound upon removal of the one or more adhesive elements.

7. The vacuum adherent dressing according to claim 1 further comprising a porous dressing material for positioning on or in the wound.

8. The vacuum adherent dressing according to claim 7 wherein the porous dressing material is at least one of a sponge or a gauze.

9. The vacuum adherent dressing according to claim 7 wherein the porous dressing material is disposed below the fluid connector under the bottom surface of the membrane.

10. The vacuum adherent dressing according to claim 1 wherein the one or more adhesive elements is secured to the skin surface, the one or more skin attachment elements having upper surfaces on which fastener portions reside that extend outward from the skin surface and matching fastener portions attached to the periphery of the membrane such that the fastener portions and the matching fastener portions are mateable to hold the membrane in position over the wound.

11. The vacuum adherent dressing according to claim 1 further comprising a portable vacuum chamber secured to the fluid connector.

12. The vacuum adherent dressing according to claim 11 wherein the portable vacuum chamber comprises at least one of a spring loaded vacuum chamber or a pump secured to the membrane.

13. The vacuum adherent dressing according to claim 1 wherein the vacuum adherent dressing is prefabricated.

14. A prefabricated vacuum adherent dressing for wound therapy of a closed wound having wound edges, the vacuum adherent dressing comprising:
(a) a flexible membrane having a top surface, a bottom surface, and a periphery, the membrane configured to extend over the wound with the periphery of the membrane being distal from the wound edges;
(b) a fluid connector provided on the membrane configured for attachment of a vacuum suction;
(d) a porous dressing material disposed below the fluid connector under the bottom surface of the membrane;
(e) an adhesive provided on the membrane; and
(f) a vacuum chamber operably connected to the fluid connector and secured to the membrane.

15. A vacuum adherent dressing according to claim 14 further comprising sealing jelly disposed below the bottom surface of the membrane, the sealing jelly configured to provide a seal between the membrane and a skin surface surrounding the wound up to and including the wound edges and wherein the periphery of the membrane is at a distance from the wound edge to create a vacuum seal between the skin surface surrounding the wound and sealing jelly without use of an adhesive.

16. The vacuum adherent dressing according to claim 15 further comprising sterile packaging that protects the prefabricated vacuum adherent dressing, the sterile packaging including a backing that extends over the sealing jelly.

17. The vacuum adherent dressing according to claim 15 wherein the adhesive comprises one or more adhesive elements at one or more locations around the periphery of the membrane for positioning the membrane over the wound.

18. The vacuum adherent dressing according to claim 17 wherein the one or more adhesive elements is secured to the skin surface, the one or more adhesive elements having upper surfaces on which fastener portions reside that extend outward from the skin surface and matching fastener portions attached to the periphery of the membrane such that the fastener portions and the matching fastener portions are mateable to hold the membrane in position over the wound.

19. A method for vacuum adherent wound therapy comprising the steps of:
(a) providing a flexible plastic membrane having a bottom surface and a top surface and a fluid connector provided on the membrane;
(b) providing sealing jelly on the bottom surface of the plastic membrane;
(c) applying the flexible plastic membrane on a skin surface having a wound therein so that the sealing jelly creates a seal between the plastic membrane and the skin surface; and
(d) applying suction to the fluid connector in order to apply therapy to the wound.

20. The method for vacuum adherent wound therapy according to claim 18 including providing a plurality of adhesive elements on a periphery of the plastic membrane to hold the plastic membrane over the wound.

21. The method for vacuum adherent wound therapy according to claim 6 including applying a porous dressing material to the wound before the flexible plastic membrane is laid over the top of the wound and a vacuum applied to the fluid connector on the top surface of the plastic membrane.

22. The method for vacuum adherent wound therapy according to claim 18 comprising applying a vacuum to at least one of an open wound or a closed wound.

23. A vacuum adherent dressing kit system for wound therapy of a wound having wound edges, the kit system comprising:
(a) a plurality of prefabricated vacuum adherent dressings comprising:
(i) a flexible membrane having a top surface, a bottom surface, and a periphery, the membrane configured to extend over the wound with the periphery of the membrane being distal from the wound edges;
(ii) a fluid connector provided on the membrane configured for attachment of a vacuum suction;
(iii) sealing jelly disposed on the bottom surface of the membrane, the sealing jelly configured to provide a seal between the membrane and a skin surface surrounding the wound up to and including the wound edges; and
(iv) adhesive elements around the periphery of the membrane for positioning the membrane over the wound;
(b) at least one package of supplemental adhesive elements for use as needed to facilitate positioning of a vacuum adherent dressing; and
(c) at least one package of supplemental sealing jelly for use as needed to facilitate positioning of a vacuum adherent dressing.

24. A vacuum adherent dressing kit system according to claim 23, wherein at least one of the vacuum adherent dressings further comprising a porous dressing material disposed below the fluid connector on a portion of the sealing jelly under the bottom surface of the membrane.

25. A vacuum adherent dressing kit system according to claim 23, further comprising at least one spring loaded vacuum chamber operably connectable to the fluid connector and securable to the membrane of a vacuum adherent dressing.
